(19) United States
(12) Patent Application Publication
(10) Pub. No.: US 2011/0071568 A1
(43) Pub. Date: Mar. 24, 2011

(54) Spacer Devices Having Retainers And Systems For The Treatment Of Spinal Stenosis And Methods For Using The Same

(76) Inventors: Richard S. Ginn, Gilroy, CA (US); Robert Elliott DeCou, San Jose, CA (US); Nicanor Domingo, Santa Clara, CA (US); Hans F. Valencia, Campbell, CA (US); David A. White, Morgan Hill, CA (US); Scott Yerby, Montara, CA (US)

(21) Appl. No.: 12/567,567

(22) Filed: Sep. 25, 2009

Related U.S. Application Data
(60) Provisional application No. 61/245,568, filed on Sep. 24, 2009.

(51) Int. Cl. A61B 17/70

(52) U.S. Cl. 606/249

(57) ABSTRACT

Spacer devices for treating spinal stenosis are provided herein, as are methods for using the same. In some example embodiments, these devices are configured for attachment over or through the interspinous ligaments. These devices generally include a spacer portion and an attachable retainer. Also provided are systems for the delivery of the spacer devices and methods for using the same.

### CROSS-REFERENCE TO RELATED APPLICATIONS

This application claims priority to U.S. Provisional Patent Application Ser. No. 61/245,568, bearing the same title and filed Sep. 24, 2009, the specification and claims of which are fully incorporated by reference herein for all purposes.

### FIELD OF THE INVENTION

The subject matter described herein relates generally to the treatment of spinal stenosis and more particularly, to interspinous spacer devices and systems for the implantation of those devices and methods for using both.

### BACKGROUND

Spinal stenosis is a condition in which a narrowing of the spinal canal and/or neural foramen leads to compression of the surrounding spinal tissue which can include the spinal cord or spinal nerves. Spinal stenosis can be caused by a number of factors, but is most commonly attributed to the natural process of spinal degeneration that occurs with aging. It has also been attributed to causes such as spinal disc herniation, osteoporosis or the presence of a tumor.

Spinal stenosis can occur locally or globally anywhere along the spinal column. When limited to a local region, spinal stenosis is most commonly found in the lumbar region and, to a lesser extent, in the cervical region. Spinal stenosis can result in numerous symptoms that are generally dependent upon the location along the spine in which the stenosis occurs. For instance, cervical spinal stenosis can result in spastic gait, numbness or weakness in upper and/or lower extremities, radicular pain in the upper limbs as well as various other muscular, intestinal and/or nervous system abnormalities. Lumbar spinal stenosis typically results in lower back pain as well as pain or abnormal sensations in the legs, thighs or feet, as well as some intestinal and/or nervous system abnormalities.

Treatment for spinal stenosis generally seeks to create additional space for the affected nerves by removing surrounding tissue or bone and/or distracting the adjacent vertebral bodies, thereby relieving the nerve compression causing the patient's symptoms. Treatment can vary from complicated surgical procedures (e.g., laminectomy and/or foraminotomy in the lumbar region, and laminectomy, hemilaminectomy and/or decompression in the cervical region), to the rigid fixation of adjacent vertebral bodies in relation to each other (e.g., spinal fusion), to the implantation of interspinous spacer devices that distract affected vertebrae without rigid fixation.

Of these, the implantation of an interspinous spacer is an attractive option for the patient since the surgical implantation procedure is relatively less invasive than spinal fusion and the patient retains more freedom in movement. Many spacer devices proposed or offered to date suffer from an over-invasive implantation procedure requiring large incisions in the back and the creation of a wide access opening to allow significant manipulations of the device to occur on the lateral side of the spinal column, or they suffer from a complicated design that does not lend itself to ease of implantation.

Furthermore, some spacer devices require dissection of the supraspinous ligament to grant access to the interspinous space and then total resection of the interspinous ligament and any spinous process overgrowth to create a cavity in which the device can be implanted. This is further to the displacement and modification of surrounding soft tissue.

Accordingly, improved interspinous spacer devices that can avoid these and other deficiencies are needed.

### SUMMARY

Example embodiments of interspinous spacer devices, delivery devices, and methods for using the same are described herein. In brief, these spacer devices generally include a spacer portion configured for placement over or through the interspinous ligament, and an attachable retainer having a bail-like configuration that encompasses and accommodates the intervening supraspinous ligament, as well as other tissue. The spacer portion can have single or multi-piece constructions. The multi-piece spacer construction can have separate elements for applying against the interspinous ligament on opposite sides, held together by the clamping force of the retainer. These elements can also pierce through the interspinous ligament and join with the opposing element, to provide a spacer with increased stability and resistance to spinal compression. Planar stabilizers can be placed on the spacer portion and/or the retainer, to stabilize the device against the superiorly and/or inferiorly located spinous processes.

Other systems, methods, features and advantages will be or will become apparent to one with skill in the art upon examination of the description herein. It is intended that all such additional systems, methods, features and advantages be included within this description, be within the scope of the subject matter described herein, and be protected by the accompanying claims. In no way should the features of the example embodiments be construed as limiting the appended claims absent express recitation of those features in the claims.

### BRIEF DESCRIPTION OF THE FIGURES

The details of the systems, devices and methods may be gleaned in part by study of the accompanying figures, in which like reference numerals refer to like parts. The components in the figures are not necessarily to scale, emphasis instead being placed upon illustrating the relevant principles. Moreover, all illustrations are intended to convey concepts, where relative sizes, shapes and other detailed attributes may be illustrated schematically rather than literally or precisely.

FIG. 1A is a perspective side view of a spinal column.

FIG. 1B is a side view of three lumbar vertebrae of a spinal column.

FIG. 1C is a top down view of a lumbar vertebral body.

FIGS. 2A-C are a perspective, top and side view, respectively, depicting an example embodiment of an interspinous spacer in an unassembled state.

FIGS. 2D-F are a perspective, top and side view, respectively, depicting the same embodiment of the interspinous spacer in an assembled state.

FIG. 2G is a side view depicting the same embodiment of the interspinous spacer implanted along a patient's spinal column.

FIG. 3A is an exploded perspective view depicting another example embodiment of an interspinous spacer in an unassembled state.

FIG. 3B is a perspective view depicting the example embodiment of the interspinous spacer in a partially assembled state.

FIG. 3C is a perspective view depicting another example embodiment of an interspinous spacer in an unassembled state.

FIGS. 3D-E are cross-sectional top views depicting additional example embodiments of an interspinous spacer in assembled states.

FIG. 3F is a perspective view depicting another example embodiment of spacer elements in an unassembled state.

FIG. 3G is a perspective view depicting another example embodiment of spacer elements in an unassembled state.

FIGS. 3H-I are top and perspective views, respectively, depicting the example embodiment of the interspinous spacer in a partially assembled state without a retainer.

FIG. 3J is a perspective view depicting another example embodiment of an interspinous spacer in an unassembled state.

FIG. 4A is a perspective view depicting another example embodiment of an interspinous spacer in an unassembled state.

FIGS. 4B-C are top views depicting the example embodiment of the interspinous spacer in various states of assembly.

FIGS. 5A-B are top and perspective views, respectively, depicting an example embodiment of a retainer.

FIG. 5C is a cross-sectional top view depicting another example embodiment of an interspinous spacer in an assembled state.

FIG. 5D is a perspective view depicting another example embodiment of an interspinous spacer in an assembled state.

FIGS. 6A-C are top, side and perspective views, respectively, depicting an example embodiment of a retainer in an at-rest state.

FIG. 6D is a perspective view depicting another example embodiment of a retainer in an open state.

FIGS. 7A-B are perspective views depicting an example embodiment of a delivery device.

### DETAILED DESCRIPTION

The present application is related to U.S. provisional patent application Ser. Nos. 61/045,169, filed Apr. 15, 2008 and 61/144,070, filed Jan. 12, 2009, and U.S. patent application Ser. No. 12/352,796, filed Jan. 13, 2009, the disclosures of which are fully incorporated by reference herein for all purposes. For example, the descriptions of the U-shaped and multi-piece spacer devices in those applications can be relevant to the spacer devices described herein, as can the description of the corresponding delivery devices and related tools, as well as the methods for using each (e.g., implantation, delivery, etc.).

The interspinous spacer devices described herein include a spacer portion that is configured to receive and couple with a retainer. The spacer portion can be configured for placement in a location between adjacent spinous processes, preferably over or through the interspinous ligament that typically exists in the span between these processes. The spacer portion is a rigid, or substantially rigid, device that can maintain a minimal spacing between adjacent spinous processes, which in turn maintains a minimum spacing for the spinal nerves thereby avoiding compression of those nerves, which can cause pain or discomfort to the patient.

The retainer preferably accommodates the presence of the supraspinous ligament and is preferably configured with a linear/curved U-shape that extends posteriorly from the spacer portion along both sides of the interspinous ligament and around the entirety of the supraspinous ligament. The retainer maintains the spacer portion in the proper orientation and position with respect to the superior and inferior spinous processes and can prevent the spacer portion from moving anteriorly towards the ligamentum flavum and spinal nerves. Implantation of the interspinous spacer devices can therefore avoiding substantial irritation or trauma to the supraspinous ligament and the anteriorly located ligamentum flavum. With a multi-piece spacer portion, the retainer can further apply a clamping force to hold the separate pieces together between the adjacent processes.

Also described herein are systems for the delivery of interspinous spacer devices for use by the administering physician or medical professional. In addition, methods for the use of the spacer devices and delivery systems are provided. These devices, systems and methods will be described herein the context of treatment of spinal stenosis in the lumbar region of the spine, although, it should be noted that these devices, systems and methods can be used to treat spinal stenosis at any location (e.g., cervical, thoracic) along the spinal column.

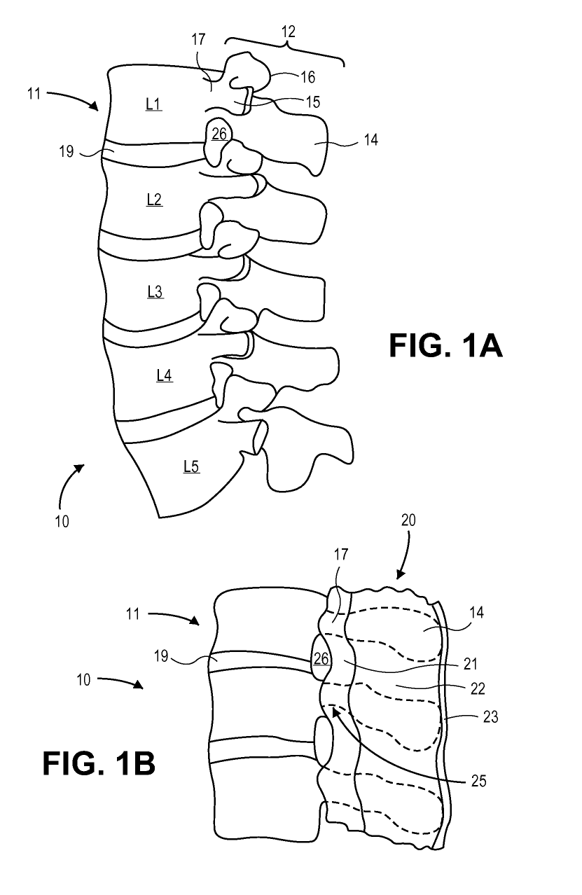

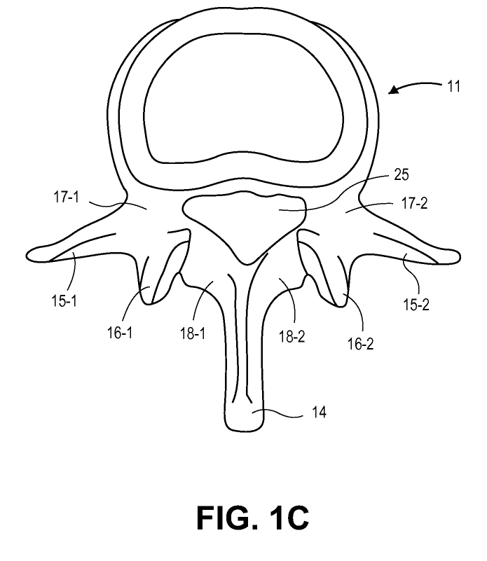

To better illustrate these devices, systems and methods, a description of the basic spinal anatomy will first be set forth. FIG. 1A is a perspective side view of a spinal column 10 showing five vertebral bodies 11, each separated by an intervertebral disc 19. More specifically, this region is the lumbar region of the spine and the five vertebral bodies 11 are lumbar vertebrae L1-L5. Each vertebral body 11 includes a posterior portion 12 having numerous bony features. The most prominent feature is spinous process 14, which is an elongate, fin-shaped feature that is situated the furthest posteriorly from each vertebral body 11. Located adjacent to spinous process 14 are left and right transverse processes 15 and left and right mamillary processes 16 (only the left side is shown here). These processes 14-16 are connected to each vertebral body 11 by way of left and right pedicles 17 (only left side shown).

FIG. 1B is a side view of three lumbar vertebrae of spinal column 10 with the left side pedicles 17 and processes 15-16 omitted to allow depiction of the interspinous tissue 20. Located adjacent to each vertebral body 11 and generally anterior to spinous process 14 (indicated as being obscured by dashed lines) is ligamentum flavum 21, which is immediately adjacent to the vertebral forman 25 and intervertebral foramen 26. Posterior to ligamentum flavum 21, is the wider interspinous ligament 22, which extends alongside each spinous process 14. Posterior to interspinous ligament 22 is supraspinous ligament 23, which generally extends along the posterior edge of the interspinous tissue 20.

FIG. 1C is a top down view of a lumbar vertebral body 11. Here, left and right pedicles 17-1 and 17-2 can be seen in greater detail extending away from vertebral body 11. Also shown is spinous process 14, left and right transverse processes 15-1 and 15-2, mamillary processes 16-1 and 16-2 and left and right lamina 18-1 and 18-2. Between features 14-18 and the bulk of vertebral body 11 is a space referred to as the vertebral foramen 25. It is through the vertebral foramen 25 and intervertebral foramen 26 (shown in FIGS. 1A-B) that the spinal cord and other spinal nerves (not shown) are routed. Spinal stenosis is generally a narrowing or reduction in size of either or both of forarnen 25-26 that results in the undesired compression of the nerves located therein.

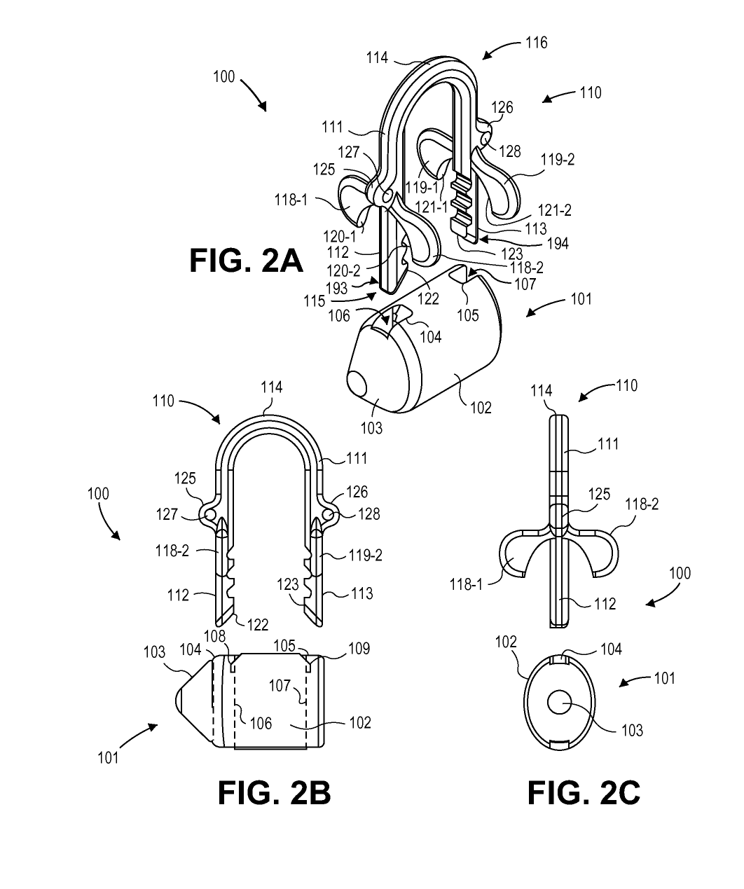

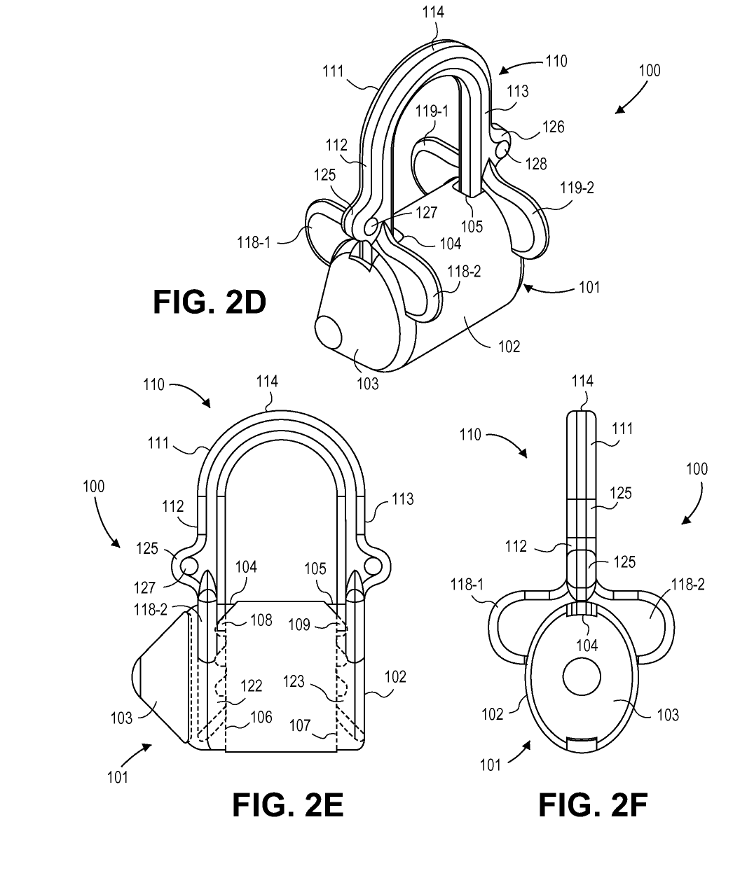

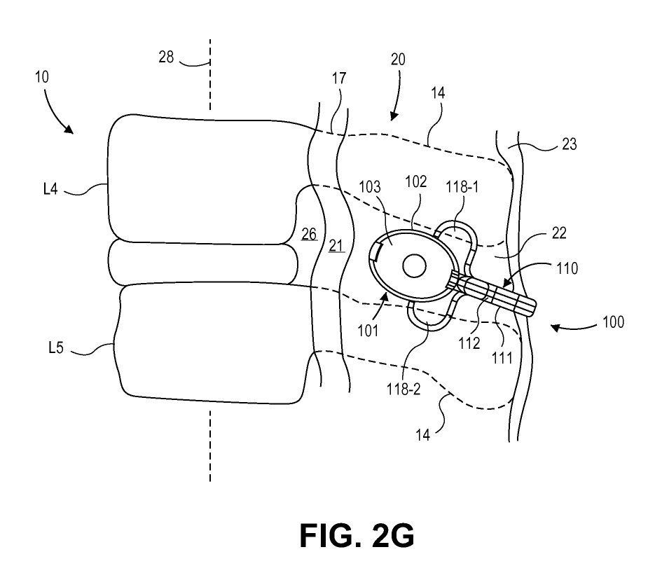

Turning now to the example embodiments, FIGS. 2A-F depict an example embodiment of a interspinous spacer device 100 configured for implantation within a patient. FIG. 2A is a perspective view depicting device 100 in an unassembled state, while FIG. 2B is a top view and FIG. 2C is a side view of device 100 in the same state. Here, device 100 includes spacer portion 101 and retainer 110, which are configured to releasably couple together. Spacer portion 101 can be configured in numerous ways, and here it is a one-piece, generally cylindrical body 102. Spacer body 102 has a generally conical end (e.g., a bullet nose) 103. Nose 103 facilitates insertion of spacer body 102 into the interspinous space (as described later). Spacer body 101 also includes openings or slots 104 and 105, which provide access to interior spaces, or channels, 106 and 107, respectively. Channels 106 and 107 are configured to receive retainer 110.

Retainer 110 can also be configured in numerous ways, and is here configured as a one-piece, generally U-shaped, or bail-like body 111 having a distal end 115 and a proximal end 116. Retainer 110 includes two elongate struts 112 and 113 connected together by a curved intermediate connective portion 114 located at proximal end 116. Retainer 110 can also be configured with more than two struts interfacing with spacer portion 101. Also, spacer device 100 can include multiple retainers 110 for interfacing with any number of spacer portions 101, or sub-bodies of spacer portion 101 (such as spacer elements 131 and 132 described later).

The free ends 193 and 194 of elongate struts 112 and 113 are tapered and configured for insertion into channels 106 and 107, respectively, to adjustably lock spacer portion 101 with retainer 110. Struts 112 and 113 can include one or more locking features 122 and 123, which are here configured as ratchet-like teeth, or abutments, respectively. These preferably each interface with locking features 108 and 109, positioned within channels 106 and 107, respectively. Here, locking features 108 and 109 are configured as catches. The distal face of each tooth 122 and 123 is preferably at approximately 45 degrees and matches the angle of the proximal face of respective catches 108 and 109. The proximal face of each tooth 122 and 123 is preferably at approximately 90 degrees and matches the angle of the distal face of respective catches 108 and 109, to lock or secure retainer 110 once engaged. Also, teeth 122 and 123 can be placed in the same positions along the length of struts 112 and 113, respectively, or can be offset.

In one example embodiment of assembly, continued advancement of retainer 110 into channels 106 and 107 causes struts 112 and 113 to deflect outwards as each successive tooth 122 and 123 transitions along the respective catch 108 and 109. Once the tooth passes the respective catch, struts 112 and 113 deflect back towards one another and engage the catch, thereby locking retainer 110 in place in the desired position. In another example embodiment, struts 112 and 113 can be deflected apart, then advanced into position and released, to allow engagement between the teeth and the respective catches.

This multi-tooth configuration allows several retainer depths for varying anatomy. If channels 106 and 107 enclose (or surround) struts 112 and 113, then adequate space should be left to allow struts 112 and 113 to deflect during advancement. Channels 106 and 107 can also have an open side along their length, to provide room for the deflection of struts 112 and 113, respectively, and also to facilitate release should it be desired. Alternatively, catches 108 and 109 can be spring-loaded so that deflection of struts 112 and 113 is not required. Interspinous spacer device 100 is shown in the assembled and locked state in corresponding FIGS. 2D-F.

One of skill in the art will readily recognize, based on this disclosure, that many other types of suitable locking devices can be used, not limited to the ratchet-type mechanism and locking features described here. For instance, clip-based, screw-based, snap-based, and high friction-based interfaces can also be used, as well as magnetic elements. Also, when spacer body 102 is singular, a locking mechanism can be provided between only one strut and the spacer body.

Struts 112 and 113 of retainer 110 also include opposing stabilizer members, which are configured here as planar lobes. Strut 112 includes opposing lobes 118-1 and 118-2, and strut 113 includes opposing lobes 119-1 and 119-2. The opposing lobes each project away from the other in an orientation that allows them to lie alongside the interspinous tissue (e.g., the interspinous ligament) and spinous processes such as depicted in FIG. 2G, and thereby provide stabilization to the device. The struts 112 and 113 of retainer 110 are generally co-planar, i.e., they reside in the same plane. Lobes 118 generally lie in the same plane, which is transverse, and preferably perpendicular to, the plane of struts 112 and 113. The same applies to lobes 119.

Lobes 118 are preferably integrally formed with body 111, but can also be attachable. Each lobe 118 includes a shaped edge 120 complementary to the surface of spacer body 102, to allow the lobe to be positioned directly adjacent spacer body 101. Lobes 119 have similar complementary shaped edges 121. Here, the shaped edges are curved to match the generally elliptical cross-profile of spacer portion 101. Lobes 118 and 119 can be included with any embodiment described herein, and can be also or alternatively located on spacing portion 101, if desired.

Struts 112 and 113 of retainer 110 also include lateral projections 125 and 126, each having an aperture, or hole, 127 and 128, respectively, for interfacing with a removal tool that can grasp projections 125 and 126 through holes 127 and 128, respectively, and use this leverage to pull struts 112 and 113 apart to release from spacer body 101.

FIG. 2G is a side view depicting this embodiment of spacer device 100 implanted within a patient's spinal column. Spacer portion 101 is positioned between the interspinous processes of the L4 and L5 vertebrae, with retainer 110 extending posteriorly along the sides of interspinous ligament 22 and around the posterior edge of supraspinous ligament 23. Spacer portion 101 is located through the interspinous ligament 22 preferably such that it does not contact the ligamentum flavum 21. Contact with the supraspinous ligament 23 can also be minimized or avoided if desired.

To implant device 100, the medical professional preferably makes one or more incisions in the back to allow access to the tissue surrounding the spinous processes. The desired interspinous space between adjacent spinous processes is then located. An incision (or other access opening) is made through the interspinous ligament, and spacer portion 101 is inserted through the incision and into position between the spinous processes. Retainer 110 is then coupled with spacer portion 101 and locked in the desired position, such that device 100 resembles that shown in FIG. 2G.

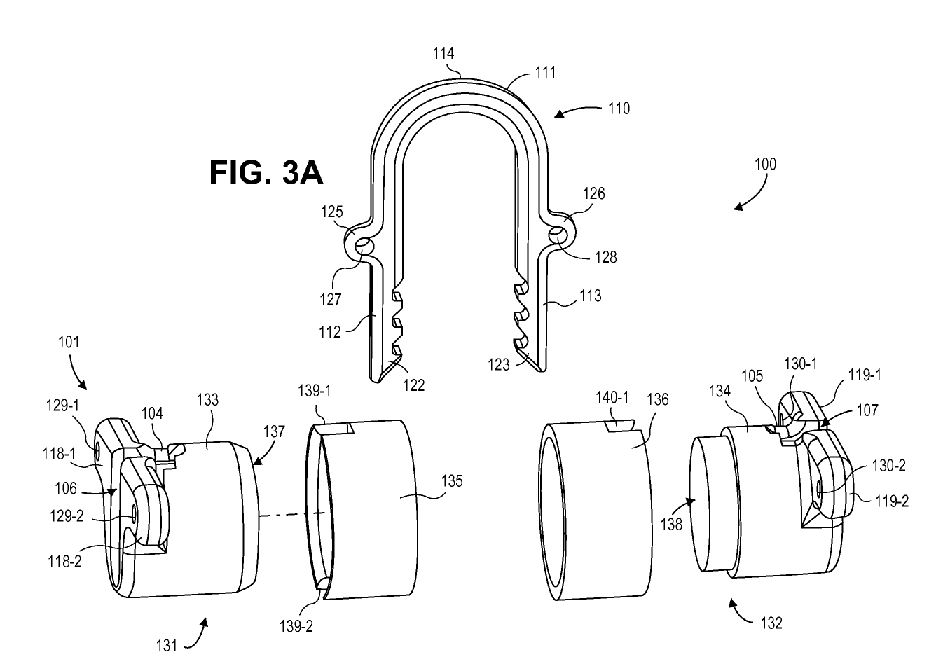

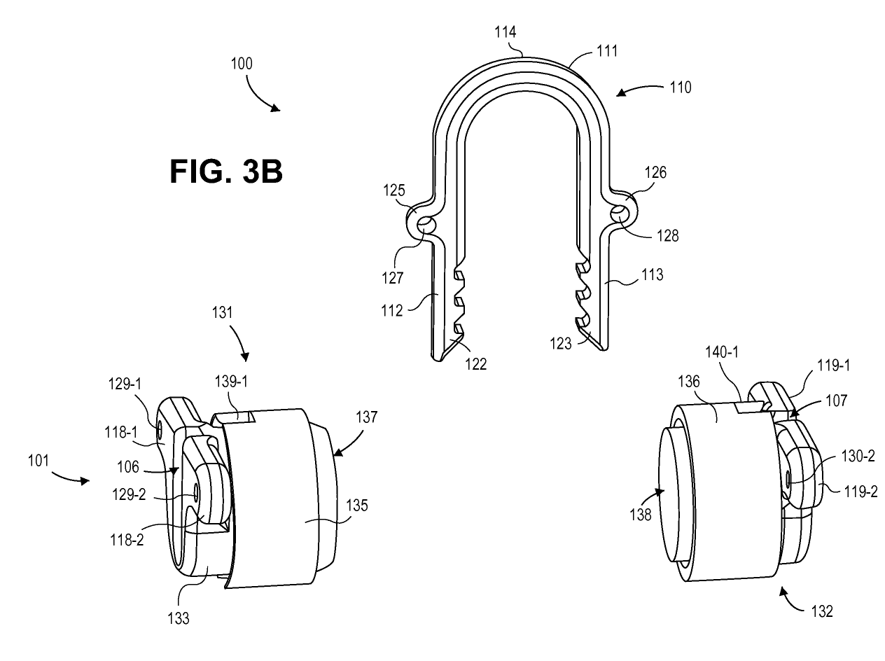

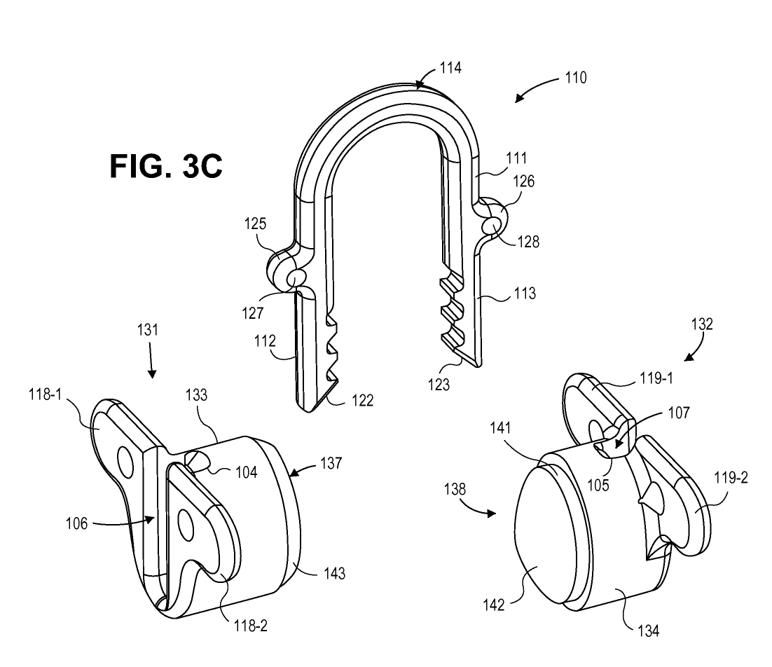

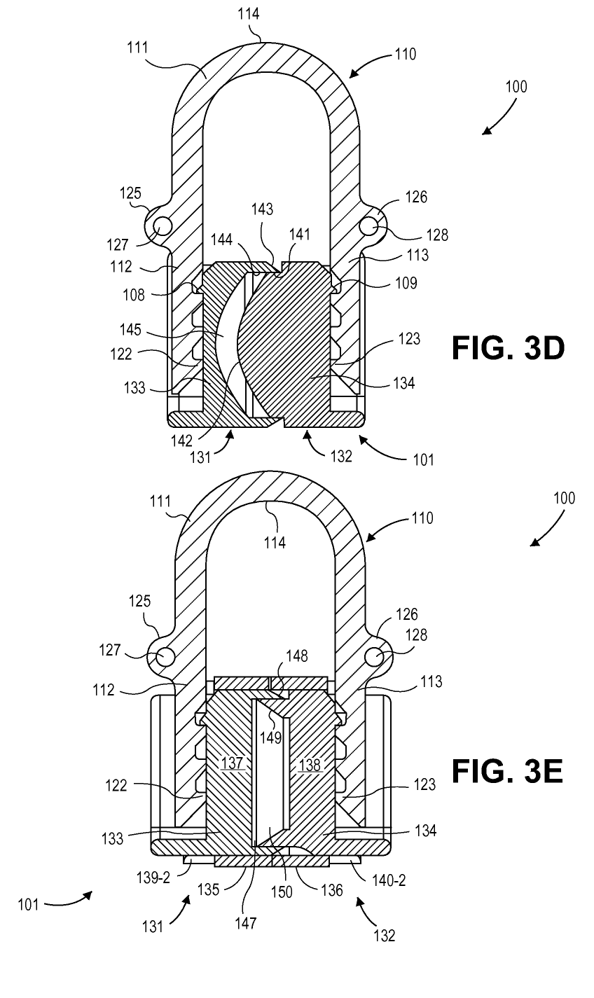

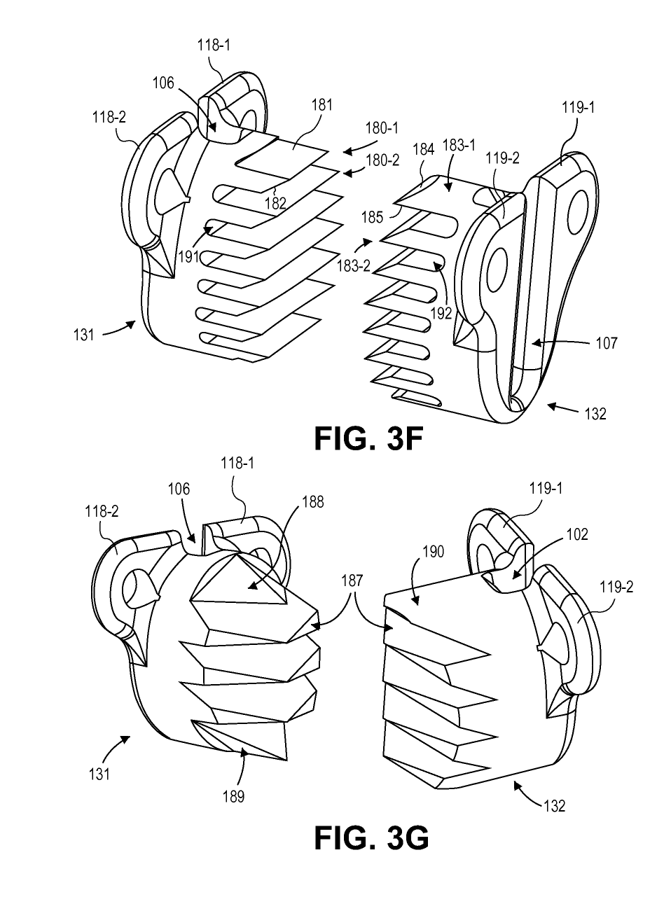

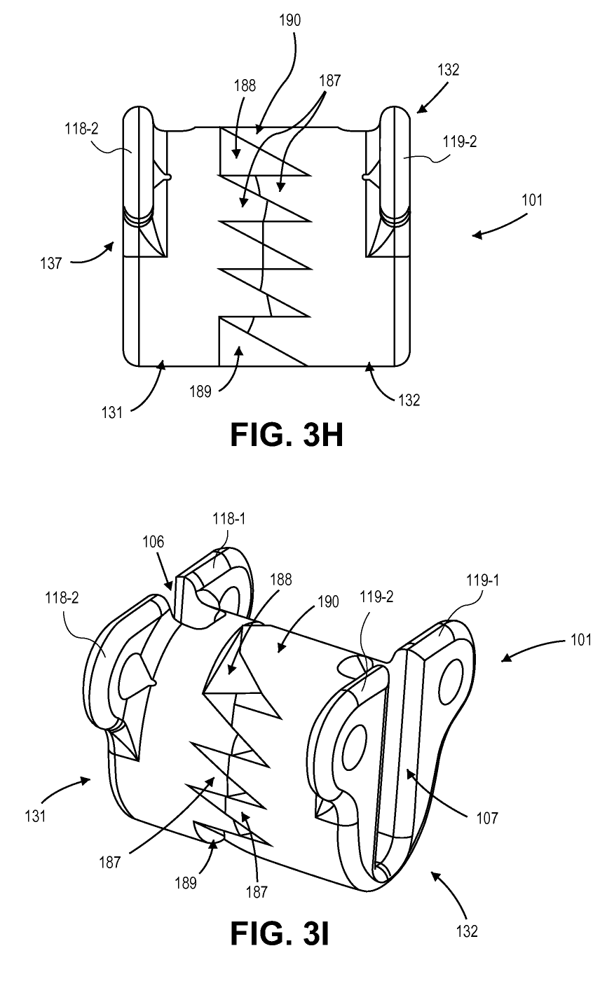

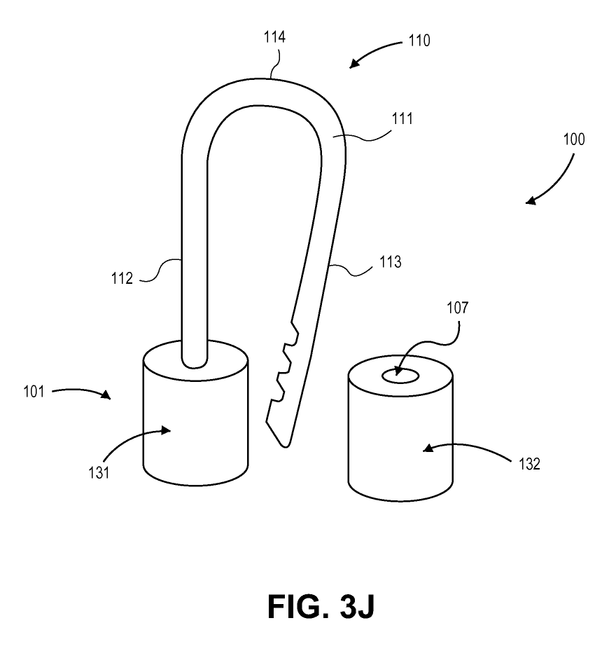

FIG. 3A is an exploded perspective view depicting another example embodiment of interspinous spacer device 100, sharing certain similarities to that of FIGS. 2A-F, although here, stabilizer members 118 and 119 are positioned on spacer portion 101, which has a multi-piece construction. The multi-piece spacer portion 101 includes inner bodies 133 and 134, each of which are configured to receive outer sleeves 135 and 136, respectively. FIG. 3B depicts sleeves 135 and 136 coupled with the inner bodies 133 and 134, respectively, to form first and second opposing spacer elements 131 and 132. More than two spacer elements can also be used, with more than two struts of retainer 110 or multiple retainers 110. Sleeve 135 has first and second openings, or slots, 139-1 and 139-2, to allow for the passage of strut 112 through channel 106. Similar slots 140 are present in sleeve 136. Sleeves 135 and 136 are generally atraumatic and formed from a softer, less rigid material to lessen any friction or impact with the adjacent tissue and bone. Sleeves 135 and 136 can be formed from a polymeric material, such as PEEK (polyetheretherketone), and the like.

Inner body 134 includes a smaller diameter cylindrical end, or nose, 138 which opposes the end 137 on inner body 133. End pieces 137 and 138 can each include opposing projecting faces, or a recessed portion, such as a cup, can be present within end 137 to receive nose 138 during implantation (i.e., to integrate or mate the space elements 131 and 132). FIG. 3C depicts another configuration without sleeves 135 and 136. Here, end 138 includes sidewall 141 and a blunt projection 142. Blunt projection 142 is configured to be received within recessed portion 145 of end 137, as depicted in the cross-sectional top view of FIG. 3D. FIG. 3D depicts retainer 110 after insertion into both of spacer elements 131 and 132. Retainer 110 is preferably made deflectable and biased towards the closed state depicted here, where spacer elements 131 and 132 are in close proximity, preferably contacting (if no intervening tissue is present, as described below). The force generated by retainer 110 is preferably sufficient to maintain spacer elements 131 and 132 in the proper position on the spinal column, as well as to resist the compressive forces generated by the superiorly and inferiorly located spinous processes, such as would occur during extension of the spine. Mating and interlocking features for the two spacer elements are described herein, and the inclusion of those features add further resistance to these compressive forces.

A blunt shape of nose 138 can aid in locating the interspinous space against which the spacer elements 131 and 132 are positioned. The medical professional can pass blunt nose 138 of spacer element 132 over the tissue and use the tactile feedback to ascertain where the adjacent spinous processes are located in relation to the interspinous space therebetween. Once the desired interspinous space is identified, spacer element 131 is placed in a position opposing spacer element 132 (if not already done so, for instance, by the delivery device). Struts 112 and 113 are deflected apart so that retainer 110 is in an open state. This allows struts 112 and 113 to then be inserted into spacer elements 131 and 132, which are separated by the interspinous tissue. Upon the locking of retainer 110 with spacer elements 131 and 132, retainer 110 is released to allow it to transition back to the closed state. Retainer 110 can also be forced anteriorly via the curved connector 114 and struts 112 and 113 will separate and return to the closed state as they pass over the catches 108 and 109. This draws or brings spacer elements 131 and 132 together into the configuration shown in FIG. 3D.

When retainer 110 closes, the interspinous tissue, which can be very thin and distensible, can be trapped between spacer elements 131 and 132. Over a period of time, this intervening trapped tissue preferably becomes necrosed and is eventually removed by the patient's own bodily processes. Apertures in the spacer elements can facilitate access to this tissue (e.g., by macrophages) to speed its removal.

If desired, these spacer elements 131 and 132 can also be configured to cut or core this intervening tissue. As shown in FIG. 3D, a close fit exists between the sidewall 141 of spacer element 132 and the inner wall 144 of spacer element 131. The leading tapered surface 143 of spacer element 131 acts as an annular, ring-like blade that incises through, or cores out a section of the interspinous ligament upon closing of the device 100.

FIG. 3E is a cross-sectional top view of another example embodiment configured to incise the intervening interspinous ligament. Here, nose 137 of spacer element 131 has a tapered outer sidewall 148 and a recessed portion 147, while nose 138 of spacer element 132 has a tapered inner sidewall 149 and a recessed portion 150. These opposing tapered edges again act to incise the intervening tissue, and trap it within recessed portions 147 and 150. This embodiment is also shown with surrounding atraumatic sleeves 135 and 136, which provide a substantially continuous surface across spacer elements 131 and 132 and cover any gaps present in the junction between the joined spacer elements 131 and 132. Although, negligible gaps may still exist, these gaps are not substantial in that they are not large enough to readily allow the adjacent spinous processes (or surrounding tissue) to begin to force the spacer elements apart during compression.

FIGS. 3F-I depict additional example embodiments of spacer elements 131 and 132 configured to cooperate to shear the intervening tissue. In the perspective view of FIG. 3F, spacer element 131 includes multiple blades 180, each having an upper flat surface 181 and a lower sloped surface 182 that meet to form a sharp edge. Spacer element 132 includes multiple blades 183, each having an upper sloped surface 184 and a lower flat surface 185 that likewise come together to form a sharp edge. The blades 180 are located in positions offset from the blades 183 of the opposing spacer element, such that the two spacer elements 131 and 132 can be brought together with the flat surfaces 181 and 185 in close proximity, or contact. These blades thus have a shearing or guillotine type effect that cuts through the intervening tissue. Sheared tissue can be displaced into recessed gaps 191 and 192 that remain present after closure, at which point the tissue can be processed naturally by the body.

FIG. 3G is a perspective view of another example embodiment of spacer elements 131 and 132 with a different blade configuration. Here, spacer element 131 includes top-most and bottom-most beveled blades 188 and 189, respectively. Beveled blade 188 has a leading, sharp end-tip and a beveled upper surface. One or more (in this case three) intervening V-shaped blades 187 are present between blades 188 and 189. V-shaped blades 187 have a leading, sharp end-tip at the junction of the upper and lower sloped surfaces. Likewise, spacer element 132 also includes V-shaped blades 187 (four) with an inverted beveled blade 190 in the top-most position. The inverted beveled blade has a surface corresponding to that of beveled blade 188, so as to receive that blade in a close fit. FIGS. 3H-I are top and perspective views, respectively, of these spacer elements joined together in a close fit.

FIG. 3J depicts another example embodiment of spacer 100 where spacer element 131 is pre-connected to strut 112 and spacer element 132 is separate. Retainer 110 is spread apart and spacer element 132 can then be connected to strut 113 during the implantation procedure. Spacer 131 and retainer 110 can be placed in the desired implantation location first with spacer element 132 attached thereafter, or conversely, spacer element 132 can be placed in the desired position first spacer element 131 and retainer 110 connected thereafter.

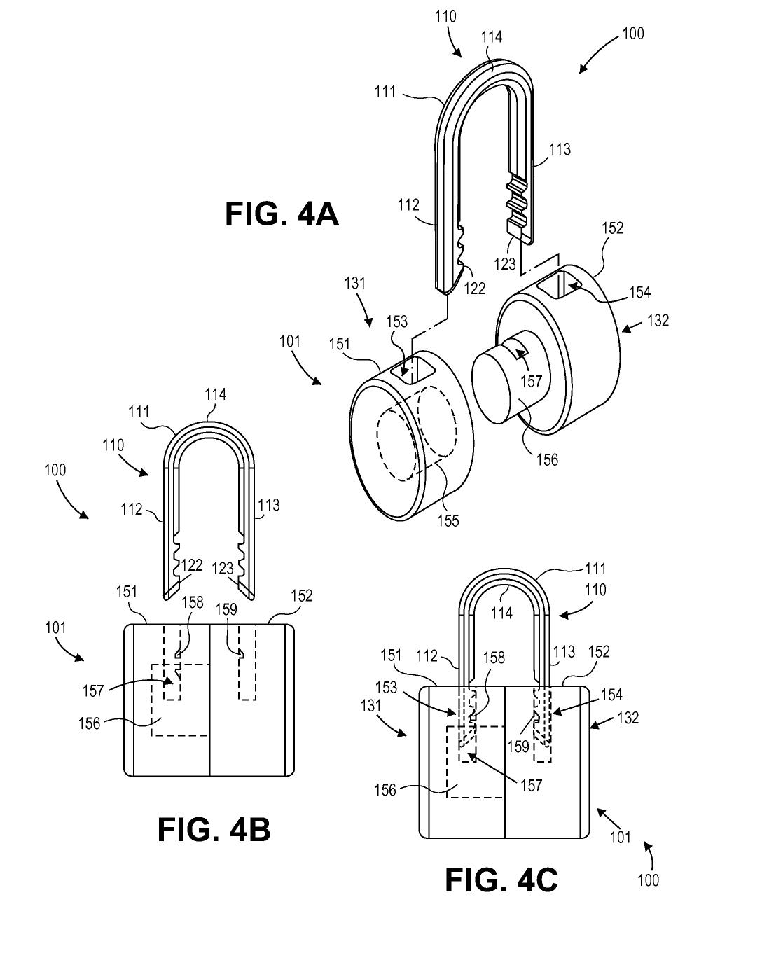

Turning now to FIGS. 4A-C, another example embodiment is depicted where spacer portion 101 includes two spacer elements 151 and 152 configured to interlock together and with retainer 110. FIG. 4A is a perspective view in an unassembled state, and FIGS. 4B-C are top views in various states of assembly. Spacer element 132 includes an extension, or hub, 156 that is insertable into a matching recess 155 in spacer element 131. Any intervening interspinous tissue is preferably removed beforehand. Spacer elements 131 and 132 include enclosed channels 153 and 154 for receiving struts 112 and 113, respectively. Hub 156 includes a sub-channel 157 that aligns with channel 153 of spacer element 131 after spacer elements 131 and 132 are inserted together. As retainer 110 is advanced into channels, teeth 122 and 123 pass over and lock with catches 158 and 159 (as seen in FIGS. 4B-C) in the selected position. FIG. 4C depicts device 100 in the first locked position and it can be seen that strut 112 extends into sub-channel 157 of extension 156 and prevents separation of spacer elements 131 and 132. The interlocking of the spacer elements 131 and 132 together, along with the retainer 110, acts to resist any tendency that those elements will separate during extension of the spine, when compressive force is imparted onto the joint between spacer elements 131 and 132 by the superior and inferior spinous processes.

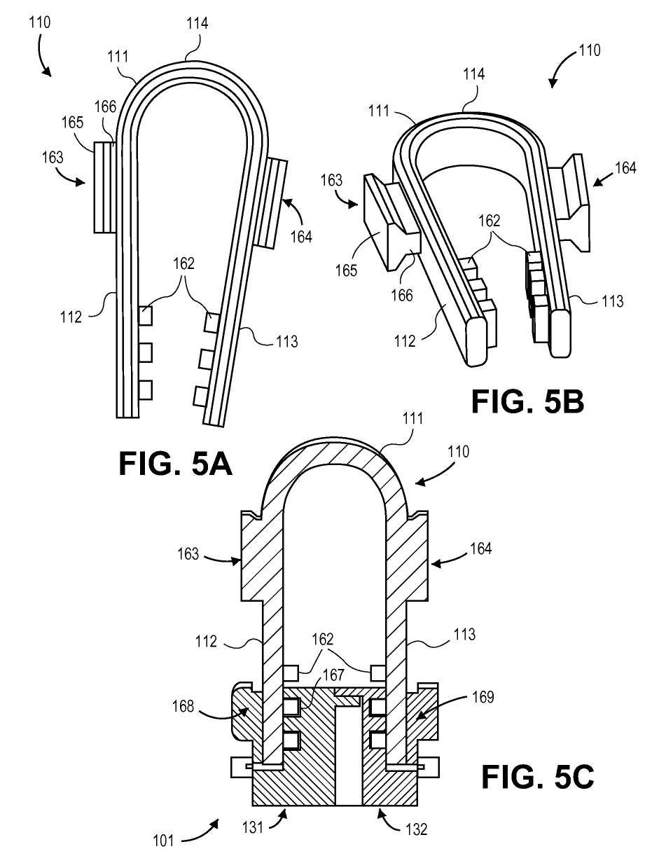

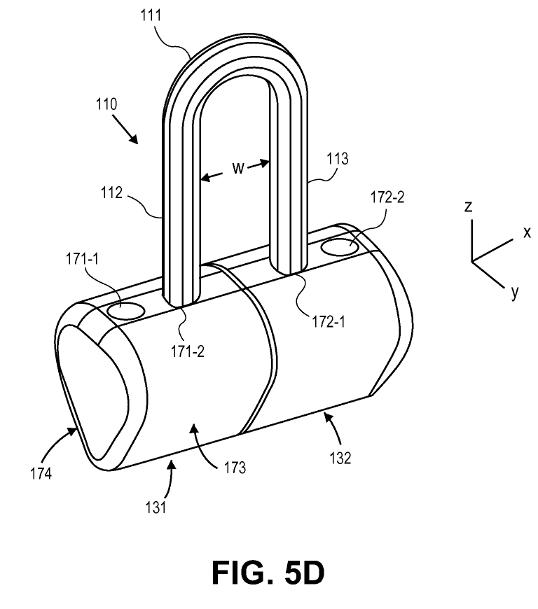

FIGS. 5A-C depict another example embodiment of spacer device 100 having a modified manner of interlocking between retainer 110 and spacer elements 131 and 132. Here, abutments (or teeth) 162 are configured as rectangular bosses, as opposed to having a sloped or non-parallel upper and lower sides. This configuration prevents movement in both the anterior and posterior directions once engaged. Any adjustment of the position of the retainer 110 requires teeth 162 to first be disengaged.

FIGS. 5A-B are top and perspective views, respectively, of retainer 110 in an at-rest state, while FIG. 5C is a cross-sectional top view depicting struts 112 and 113 of retainer 110 deflected outwards, or opened, so as to allow engagement with spacer elements 131 and 132. Spacer elements 131 and 132 have open channels 168 and 169 to allow struts 112 and 113, respectively, to be inserted such that teeth 162 engage the desired recesses 167, which preferably also have a rectangular shape. One of skill in the art will readily recognize that other shapes for teeth 162 and recesses 167 can be used that will still increase resistance to movement in both anterior and posterior directions. The bias of retainer 110 towards the at-rest state holds it in place against spacer elements 131 and 132 (even if retainer 110 is in an intermediate state and not fully transitioned into the at-rest state).

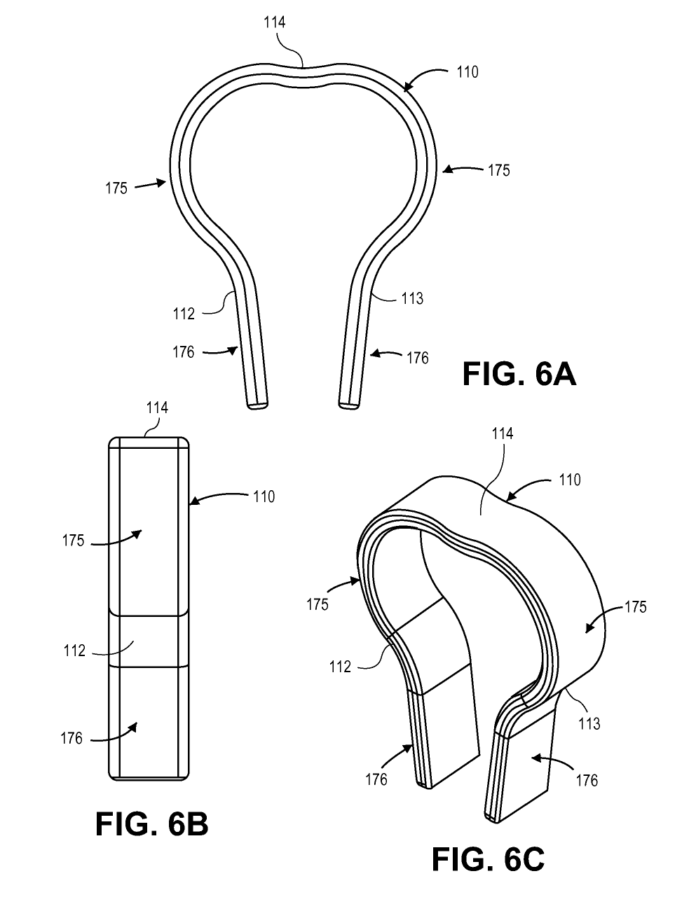

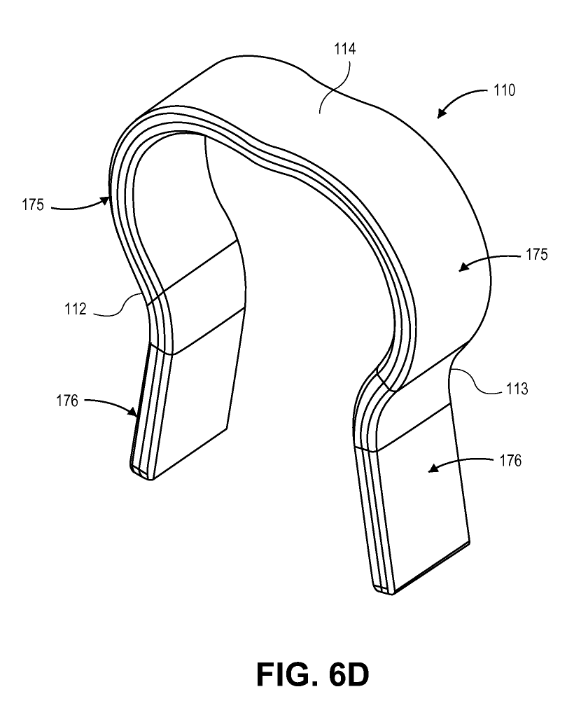

As shown in FIG. 5A, strut ends are closer to each other than in the more open state of FIG. 5C. Generally, the closer the strut ends are in the at-rest state, the more force that can be generated when in the state of FIG. 5C, so long as significant plastic deformation does not occur when spreading the strut ends. Furthermore, more closure force is generally applied when only the distal-most tooth is engaged, as opposed to the proximal-most tooth (e.g., when retainer 110 is fully advanced). Another embodiment of retainer 110 capable of applying relatively greater force at each tooth position as compared with this embodiment, is described with respect to FIGS. 6A-D below.

This embodiment of FIGS. 5A-C also includes engagement features 163 and 164 on struts 112 and 113, respectively. These features are provided to allow a delivery device to more readily grasp the struts and retract them, or maintain them in an “open” retracted state during delivery. Once in the desired position, struts 112 and 113 can be slowly released to allow retainer 110 to transition back to the at-rest state and interlock with spacer elements 131 and 132. Here, engagement features 163 and 164 are shaped in a dovetail fashion, with a narrow base, or neck, 166 and a relatively wider fan-out portion 165. This configuration can also be referred to as T-shaped.

FIG. 5D depicts another example embodiment where spacer elements 131 and 132 have multiple channels 171 and 172. The presence of multiple channels can allow customization for different anatomies. The medical professional can use different retainers 110 having varying widths W to accommodate different thicknesses in the posterior region of the patients spine. Multiple channels can also allow for interfacing with delivery or removal instrumentation. For instance, outer channels 171-1 and 172-2 can receive a spanner wrench-type instruement that can open and close spacer elements 131 and 132. Any number of channels can be included in each spacer element. Here, channels 171 and 172 are spaced along the lateral X axis, but the channels can also be spaced along the Y axis, in which case the width would remain constant but the position of retainer 110 could vary superiorly or inferiorly. Also, any combination of channels can be provided along both X and Y axes, to provide further adaptability.

Spacer elements 131 and 132 also have a tapered configuration (rounded triangular cross-sectional profile), such that sloped faces 173 and 174 come together at the anterior end of the spacer elements 131 and 132. This demonstrates the adaptability of the spacer elements to account for anatomical variations. Spacer elements 131 and 132 can have other cross-sectional profiles, such as egg-shaped, elliptical, oval, and circular, or rounded polygonal profiles such as rectangular, square, pentagonal, hexagonal, octogonal, and the like.

FIGS. 6A-D depict another example embodiment of retainer 110 configured to provide relatively greater closure force. FIG. 6A is a top view of retainer 110 in the at-rest state, while FIG. 6B is a side view and FIG. 6C is a perspective view of the same. The ratchet teeth are not shown. FIG. 6D is a perspective view showing retainer 110 in an outwardly deflected, or open, state. Each strut 112 and 113 includes a curved posterior (proximal) portion 175 and a relatively straight anterior (distal) portion 176. The width of retainer in the posterior section is relatively greater than the width in the anterior section, as the posterior section of each strut flares outwards away from the opposite strut. It should be noted that this configuration of retainer 110 can be used with any embodiment of spacer 100 described herein.

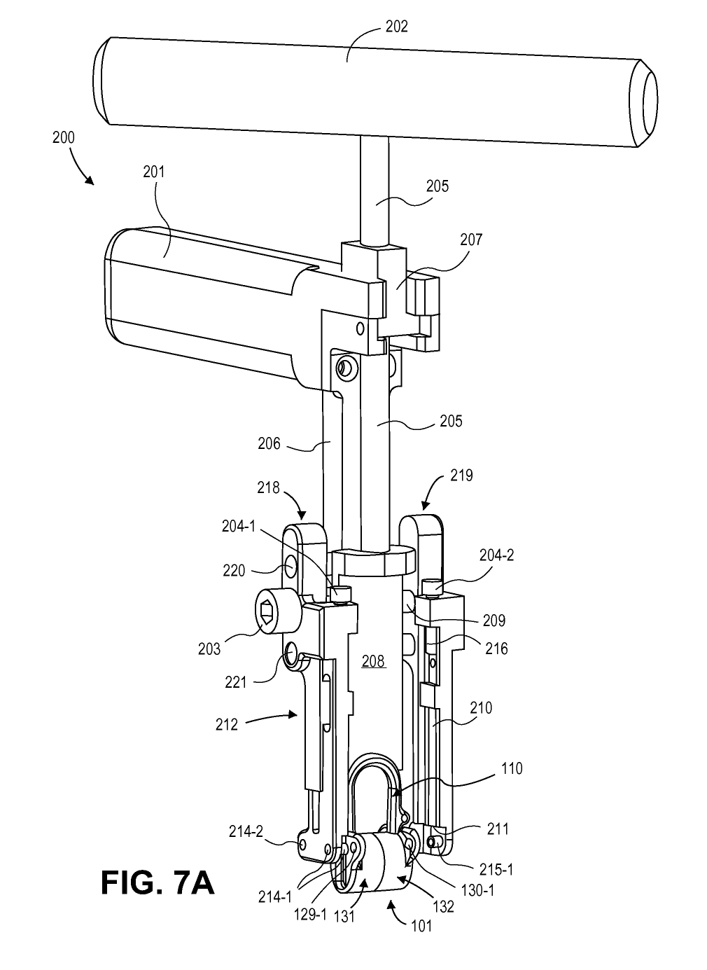

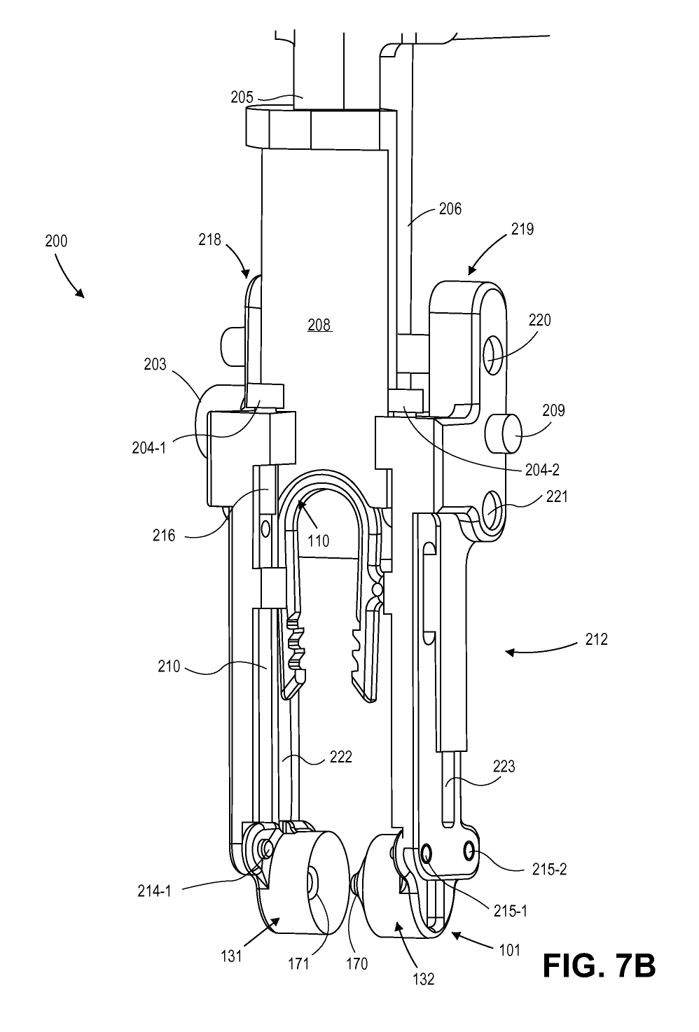

FIG. 7A is a perspective view depicting an example embodiment of delivery device 200. Here, delivery device 200 is used with an embodiment of spacer device 100 similar to that depicted in FIG. 3C. Delivery device 200 includes a main handle 201 which is connected to a housing 207 and a device shaft 206, which is in turn connected to a spacer interfacing device 212. FIG. 7B depicts spacer interfacing device 212 in greater detail, and while interfacing with another embodiment of spacer device 100, similar to that of FIG. 7A but having a projecting nipple-like feature 170 on spacer element 132 and a corresponding recess in spacer element 131, the sloped surfaces of which aid in self-alignment of elements 131 and 132 during delivery. FIG. 7B depicts spacer elements 131 and 132 (fully apart) and retainer 110 prior to engagement. FIG. 7A depicts spacer elements 131 and 132 after having been brought together and retainer 110 after engagement with spacer elements 131 and 132, i.e., near the completion of the delivery procedure.

Interfacing device 212 includes distal seats on which spacer elements 131 and 132 are placed. These seats can be configured as one or more pins 214 and 215, which are insertable into corresponding apertures 129 and 130, respectively, in spacer elements 131 and 132. Spacer elements 131 and 132 are held in place by a locking mechanism, which are slidable bars 210. Bars 210 slide within channels in the sidewalls of interfacing device 212. The position of these bars 210 is controlled by actuators 204-1 and 204-2, respectively, which are configured here as hexagonal bolts that reside within threaded lumens inside interfacing device 212. Advancement of bolts 204 cause the bolt shafts 216 to depress bars 210 and lock spacer elements 131 and 132 in place (shown in FIG. 7B). Device 200 is preferably used, in this configuration, to position spacer elements 131 and 132 appropriately, for example, by using tactile feedback provided by projection 170.

Once in the desired position, spacer elements 131 and 132 are brought together across the interspinous space. This can be accomplished with actuator 203, which is also configured as a hexagonal bolt. Tightening of actuator 203 causes threaded bolt shaft 209 to draw the right side portion 219 of interfacing device 212 towards the left side portion 218. Relative motion of side portions 218 and 219 is guided by alignment pins 220 and 221.

Delivery device 200 also includes an actuator 202, configured here as a handle, for controlling the position of retainer 110 with respect to spacer elements 131 and 132. Actuator 202 is coupled with a shaft 205, which is threaded through an axial nut within housing 207 (and thus not shown). The distal end of shaft 205 is coupled with a retainer interface 208, which is configured here as a sled. The distal end of sled 208 has a curved surface corresponding to the shape of the proximal portion of retainer 110. In this embodiment, retainer 110 is biased towards a closed configuration, but the curved receptacle of sled 208 compresses retainer 110 beyond the closed configuration such that retainer 110 is biased to expand from the configuration shown. This compression holds retainer 110 to sled 208 passively, without the need for an active (i.e., capable of opening and/or closing) retaining mechanism, although one can be provided if desired.

Rotation of actuator 202 causes sled 208 to advance distally and drive retainer 110 downwards into spacer elements 131 and 132 after closure. Side portions 218 and 219 each include a guide slot 222 and 223, respectively, for guiding the advancement of retainer 110. Once retainer 110 is engaged with spacer elements 131 and 132, actuator 203 can be reversed to spread side portions 218 and 219 apart again and release spacer device 100, as depicted in FIG. 7A.

One of skill in the art will readily recognize that the actuators of delivery device 200 can be manually controlled or electrically controlled, such as with an electronic interface. Furthermore, device 200 can include visual guides that instruct the medical professional as to the position of the spacer components and the proper delivery sequence. These guides can be printed or can be provided through an electronic display.

The components of spacer device 100 can be formed from any number or types of materials that are suitable for the needs of the individual application. Each of spacer body 102, the main (core) portions of spacer elements 131 and 132, and retainer 110 can be formed from metallic or polymeric materials. Retainer 110 is preferably (but not necessarily) formed from elastic (or superelastic) shape memory materials, i.e., materials that can exhibit a bias to revert towards a predetermined shape or state, such as nickel-titanium alloys (e.g., nitinol) and the like. This bias can be present before and after implantation or can be configured to initiate once a predetermined temperature is reached (e.g., slightly below human body temperature). Other suitable materials include titanium, stainless steel, Elgiloy and various polymers such as polyetheretherketones (PEEK), polycarbonate urethane (PCU), ultra high molecular weight polyethylene (UHMWPE), and the like. Materials that are not magnetic can allow compatibility with magnetic resonance imaging (MRI) systems. Materials that approximate bone density, such as PEEK, can minimize trauma to the adjacent spinous processes and are especially suitable for sleeves 135 and 136. Each of spacer elements 131 and 132 can also be formed from the same or different materials. Any portion or body of spacer 100 can itself be formed from any number of one (monolithic) or more (multi-body) separate pieces. For example, struts 112 and 113 can be formed from a rigid (i.e., inflexible) material and connective portion 104 can be formed from a more flexible material, for instance, to ease bending in that region or to minimize irritation to the supraspinous ligament. Alternatively, body 111 can be monolithic, as shown in the figures. Likewise, the stabilizers can be made integral with retainer 110, or spacer elements 131 and 132, or can be attached separately.

Furthermore, any portion of spacer device 100 can be coated with any desired material, such as bio-compatible substances, substances to alter the surface friction (either increase or decrease) between the device and any surrounding tissue, substances to promote healing, atraumatic and conformable substances as described earlier, absorbable and other substances to promote the growth of scar tissue or other tissue (e.g., poly-L-lactide (PLLA), polyglycolide (PGA), sheep intestinal submucosa, etc.), and the like.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Statements expressly indicating that certain features are not limited in a particular manner should not be interpreted as implying that the absence of such statements with regard to other features implies that those other features are in any way limited to the disclosed embodiment.

What is claimed is:

1. An interspinous spacer device, comprising:
a retainer comprising first and second elongate struts each with a free end, the free ends being on a distal side of the retainer and the struts being connected at a proximal side of the retainer, and
a spacer portion being securable to the free ends of the first and second struts, the spacer portion comprising a first channel configured to receive the free end of the first strut and a second channel configured to receive the free end of the second strut, wherein the spacer portion is adapted to maintain a spacing between adjacent spinous processes of a patient.

2. The spacer device of claim 1, wherein the spacer portion is a single body, having two lateral ends, a first lateral end having a general conical shape.

3. The spacer device of claim 1, wherein the spacer portion further comprises a locking feature in each channel, the locking feature configured to engage the respective strut when inserted therein.

4. The spacer device of claim 3, wherein each strut has one or more abutments configured to selectively engage with the locking feature in the respective channel.

5. The spacer device of claim 1, wherein the retainer is generally U-shaped and the struts are located in the same plane, further comprising a planar stabilizer coupled with the retainer.

6. The spacer device of claim 5, wherein the planar stabilizer is oriented on a plane perpendicular to the plane of the struts.

7. The spacer device of claim 6, wherein the planar stabilizer is a first planar stabilizer coupled with the first strut, the spacer device further comprising a second planar stabilizer coupled with the second strut, the second planar stabilizer also being oriented on a plane perpendicular to the plane of the struts, wherein the first and second planar stabilizers each include two planar stabilizer lobes.

8. The spacer device of claim 6, wherein the planar stabilizer comprises a shaped surface corresponding to the surface of the spacer portion.

9. The spacer device of claim 1, wherein the spacer portion comprises two bodies, each body being a spacer element adapted to maintain a spacing between adjacent spinous processes of a patient.

10. The spacer device of claim 9, wherein at least one of the spacer elements comprises a planar stabilizer.

11. The spacer device of claim 9, wherein the first channel is in the first spacer element and the second channel is in the second spacer element.

12. The spacer device of claim 11, wherein each channel is open along its length and located between the first and second planar stabilizers of the respective spacer element.

13. The spacer device of claim 9, wherein each spacer element comprises a planar stabilizer, the planar stabilizers of each spacer element being located on a first side of the respective spacer element, and wherein the first spacer element has a second side opposite its first side, and wherein the second spacer element has a second side opposite its first side, the second sides of the spacer elements being configured to interface with each other.

14. The spacer device of claim 9, wherein the first spacer element comprises a projection and the second spacer element comprises a recess configured to receive the projection.

15. The spacer device of claim 9, wherein at least one of the first and second spacer elements comprises a sharp edge configured to cut tissue.

16. The spacer device of claim 9, wherein the retainer has a first state where the spacing between the ends of the struts is a first distance, and the retainer being deflectable into a second state where the spacing between the ends of the struts is relatively greater, wherein the retainer is biased towards the first state.

17. The spacer element of claim 9, wherein the first spacer element comprises an elongate projection having a sub-channel, the elongate projection being receivable in a recess in the second spacer element such that the second channel of the second spacer element aligns with the sub-channel of the elongate projection.

18. The spacer device of claim 9, wherein each spacer element further comprises an outer sleeve surrounding the spacer element.

19. The spacer device of claim 1, wherein each strut has a generally straight distal portion and a generally curved proximal portion, the generally curved proximal portion flaring outwards away from the opposite strut.

20. The spacer device of claim 1, wherein the spacer portion further comprises a plurality of box-like recesses in each channel and the retainer comprises a box-like projection and a dovetail feature on each strut, the recesses of each channel being configured to engage with the box-like projection of the respective strut.
